(19) United States
(12) Patent Application Publication
(10) Pub. No.: US 2009/0076613 A1
(43) Pub. Date: Mar. 19, 2009

(54) INTERVERTEBRAL DISC PROSTHESIS

(76) Inventors: Lutz Biedermann, VS-Villingen (DE); Wilfried Matthis, Weisweil (DE); Jurgen Harms, Karlsruhe (DE)

Correspondence Address: CHRISTIE, PARKER & HALE, LLP, PO BOX 7068, PASADENA, CA, 91109-7068, US

(21) Appl. No.: 12/180,481

(22) Filed: Jul. 25, 2008

Related U.S. Application Data
(60) Provisional application No. 60/952,201, filed on Jul. 26, 2007.

(30) Foreign Application Priority Data
Jul. 26, 2007 (EP) 07014709.5

(51) Int. Cl. A61F 2/44; A61B 19/00

(52) U.S. Cl. 623/17.16; 623/17.11; 623/17.13; 128/898

(57) ABSTRACT

An intervertebral disc prosthesis includes a base plate, a top plate, a central axis extending through the center of the base plate and the top plate, and at least two springs arranged between the base plate and the top plate. The springs each have a loop-shaped section and two free ends. One of the free ends of the springs is connected to the base plate and the other one of the free ends is connected to the top plate. The loop-shaped section is directed away from the central axis.

### CROSS-REFERENCE TO RELATED APPLICATIONS

The present application claims the benefit of Provisional Patent Application Ser. No. 60/952,201, filed Jul. 26, 2007, and claims priority from European Patent Application EP 07014709.5, filed Jul. 26, 2007, the disclosures of which are incorporated herein by reference.

### BACKGROUND

The disclosure relates to an intervertebral disc prosthesis.

An intervertebral disc prosthesis using metal springs arranged between a base plate and a top plate is known from, for example, U.S. Pat. No. 4,309,777. This artificial intervertebral disc comprises an upper and a lower disc portion with a plurality of helical springs extending from the upper to the lower disc portion which yieldably urge the disc portions away from each other.

U.S. Pat. No. 6,770,094 B2 describes an intervertebral disc prosthesis comprising a cranial disc and a caudal disc, which are supported against each other elastically upon compression by springs. The springs consist of a memory-metal alloy which exhibits super-elastic properties at body temperature. In one embodiment the springs are formed by leaf springs which have the form of strips, one end of which is mounted on the upper disc and the other end of which is mounted on the lower disc.

U.S. Pat. No. 7,201,776 B2 discloses an artificial disc replacement (ADR) including a pair of opposing endplate components, each attached to one of the upper and lower vertebrae, a cushioning component disposed between the endplate components, and a filler material contained within the cushioning component. In one embodiment the cushioning component is a tire-like component which cooperates with a metal hub in the center of the device which holds the air, fluid, gel or other material within the tire.

Commercially available intervertebral disc prostheses may have one ore several disadvantages such as no axial damping, no rotational control, no restoring in all degrees of freedom and a Range of Motion (ROM) which is excessive if compared to a natural disc.

Based on the foregoing, there is a need to provide an intervertebral disc prosthesis which has improved axial damping combined with rotational control and flexion/extension control.

### SUMMARY OF THE INVENTION

According to the disclosure, an intervertebral disc prosthesis is provided which includes a base plate, a top plate opposite to the base plate, and central axis extending through the center of the base plate and the top plate and a plurality of springs each having a loop-shaped section and two free ends, the springs being arranged around the central axis, wherein the free ends are connected to the base plate and the top plate, respectively and wherein the loop-shaped section is directed away from the central axis of the prosthesis.

The intervertebral disc prosthesis according to the disclosure achieves high axial damping with loop-shaped springs having the loop portion directed away from the central axis. Since the springs are connected with their free ends to the base plate and the top plate, respectively, a rotational control is achieved. The Range of Motion (ROM) in flexion/extension is reduced to approximately +/−5° which protects the surrounding joints, in particular the facet joints.

An end stop which can be made of an elastomer material additionally restricts and damps the axial movement.

Flexible protection sleeves prevent vessels and tissue from growing into the space between the springs.

Further features and advantages of the disclosure will become apparent to those skilled in the art from the following description of the preferred embodiments of the invention which have been shown and described by way of illustration. As will be realized, the disclosure is capable of other and different embodiments, and its details are capable of modification in various respects. Accordingly, the drawings and the description are to be regarded as illustrative in nature and not as restrictive.

### BRIEF DESCRIPTION OF THE DRAWINGS

FIG. 1 shows as sectional view of an intervertebral disc prosthesis according to an embodiment of the disclosure.

FIG. 2 shows a perspective side view, partially in section of the intervertebral disc prosthesis according to FIG. 1

FIG. 3 shows a perspective exploded view of the intervertebral disc prosthesis according to FIG. 1.

FIG. 4 shows a perspective side view of the intervertebral disc prosthesis of FIG. 1.

FIG. 5 shows a portion of the intervertebral disc of FIG. 1 which is used for clamping the springs to the top plate.

### DETAILED DESCRIPTION OF THE INVENTION

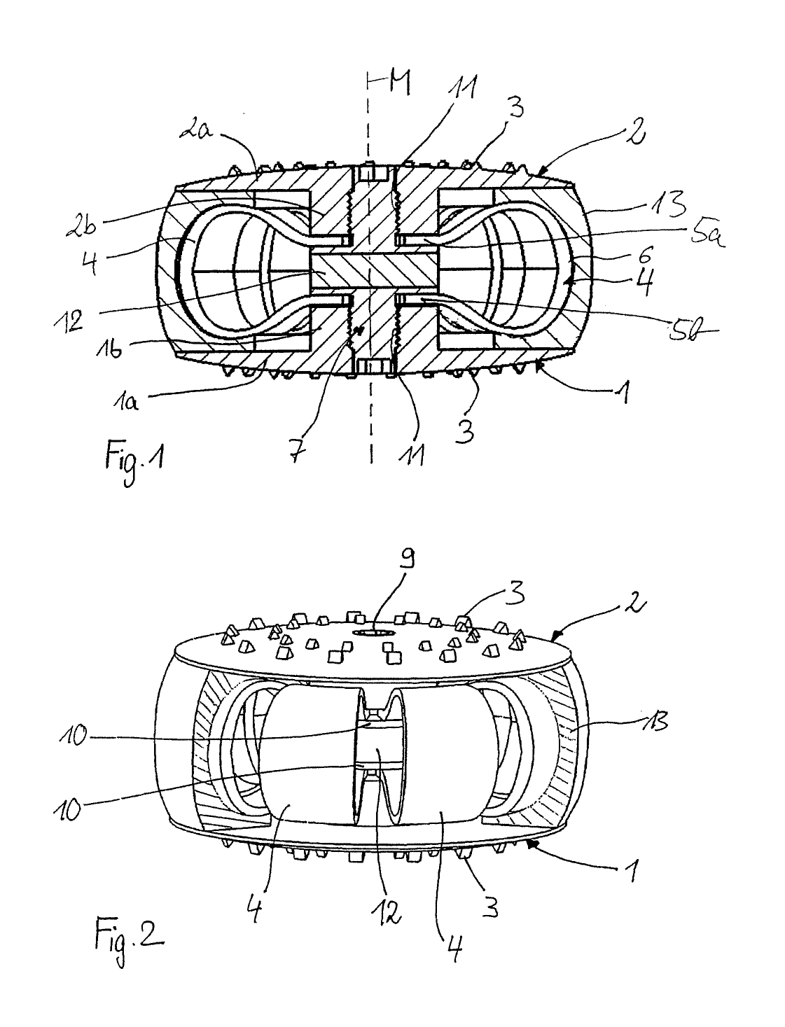

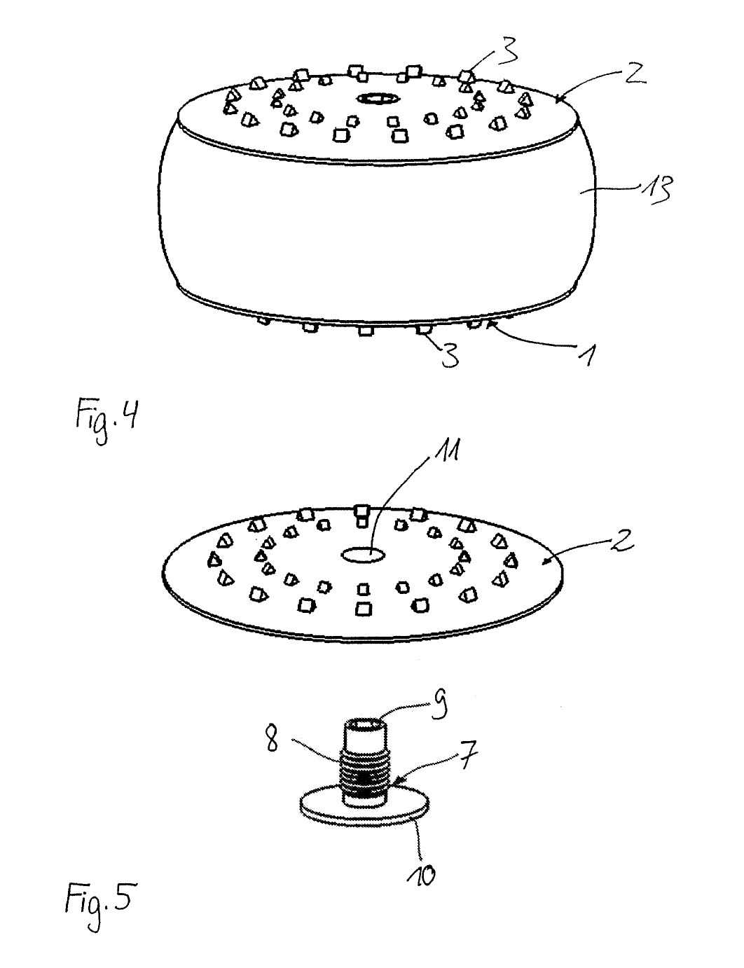

As can be seen from FIGS. 1 to 4, an intervertebral disc prosthesis includes a base plate 1 and a top plate 2 opposite to the base plate. The base plate 1 and the top plate 2 each have a plate portion 1a, 2a and a central cylindrical projection 1b,2b. The base plate 1 and the top plate 2 are arranged such that the cylindrical projection 1b, 2b are facing each other. A central axis M extends through the center of the projections 1b,2b. A threaded bore 11 extends through each of the base plate 1 and the top plate 2 which is coaxial with the cylindrical projection 1b, 2b. The plate portions 1a, 2a have in the embodiment shown a circular shape but can have any other shape, in particular a shape adapted to the contour of the end plates of the adjacent vertebrae. The plate portions 1a, 2a are slightly curved outward and each plate portion includes a plurality of teeth 3 for anchoring in the end plates of the adjacent vertebrae.

The base plate 1 and the top plate 2 are made of biocompatible material, such as titanium or stainless steel or a biocompatible plastic material such as for example polyaryletheretherketone (PEEK).

A plurality of springs 4 are arranged between the base plate 1 and the top plate 2. As can be seen in particular in FIG. 3 each spring 4 has the shape of a substantially flat strip similar to a leaf spring and comprises two free end sections 5a,5b and a loop-shaped bent section 6 between the free end sections 5a,5b. The springs can be leaf springs.

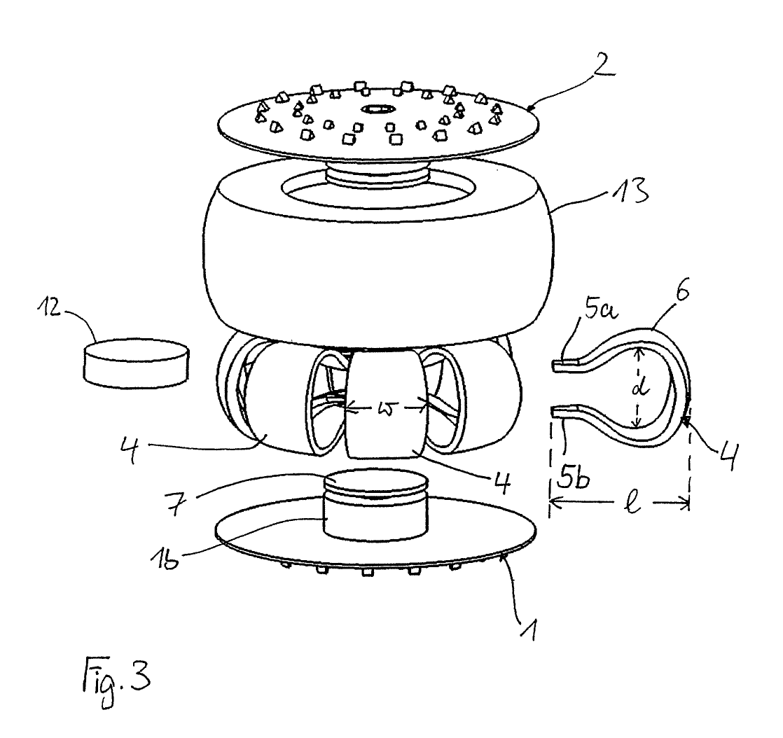

The end sections 5a,5b are straight flat sections as can be seen in particular in FIG. 1. The width w of the strip which forms the spring 4 is such that a plurality springs, for example, six springs, can be arranged around the central axis M with a space between adjacent springs. The width w is largest at the outermost part of the loop-shaped section 6 and decreases towards the end sections 5a,5b as can be seen in FIG. 3. The length 1 of the spring 4 from the end sections 5a,5b up to the outermost part of the loop-shaped section 6 is such that when the spring is mounted the loop-shaped section 6 lies completely between the plate portions 1a,2a. The largest diameter d of the loop-shaped section 6 in a direction parallel to the central axis M of the prosthesis is such that in the mounted state shown in FIGS. 1 and 2 there is a distance between the spring 4 and the base plate 1 and the top plate 2.

The springs 4 can be made of any highly flexible biocompatible material. For example, the spring can be made of a shape-memory material exhibiting super-elasticity, in particular of a shape-memory metal such as a nickel titanium alloy. A specific example for this alloy is nitinol.

As can be seen in FIGS. 1, 3 and 5, mounting screws 7 are provided for mounting the springs between the base plate 1 and the top plate 2. Each mounting screw 7 includes a threaded screw section 8 and recess 9 at the free end thereof for engagement with a screwing-in tool. Opposite to the free end the mounting screw 7 includes a disc-shaped portion 10 the diameter of which corresponds to the diameter of the cylindrical projection 1b,2b. Each screw section 8 engages with the coaxial threaded bore 11 extending through the base plate 1 and its cylindrical projection 1b and through the top plate 2 and its cylindrical projection 2b, respectively. The length of the screw section 8 is such that when the mounting screws 7 are connected to the base plate 1 and the top plate 2, respectively, and the spring 4 is clamped by the disc-shaped portion 10 and the cylindrical projection 1b, 2b, the recess 9 can be engaged with the screwing-in tool. The mounting screws 7 are made of a biocompatible material like the base plate and the top plate.

Between the disc-shaped portions 10 of the mounting screws 7 a damping element 12 is provided. The damping element can be shaped as a disc and made of an elastomer material such as polycarbonate uretane (PCU). The thickness of the disc is selected such that it fills the space between the mounting screws 7. The material is selected such that the desired damping effect can be achieved.

As can be seen in FIGS. 1 to 4, a protective sleeve 13 is provided which surrounds the springs 4 circumferentially and extends from the base plate 1 to the top plate 2. The protective sleeve 13 is made of a flexible material, in particular it can be made of an elastomer material such as polycarbonate uretane (PCU). In a radial direction, the sleeve 13 partially encompasses the loop-shaped sections 6 of the springs 4. Hence, the loop-shaped sections 6 are not in direct contact with the plate portions 1a, 2a of the base plate 1 and the top plate 2. This leads to a further axial damping.

The intervertebral disc prosthesis is pre-assembled in such a way that the springs 4 are clamped between the disc-shaped portions 10 of the mounting screws 7 and the cylindrical projections 1b, 2b of the base plate 1 and the top plate 2, respectively. The damping element 12 may be fixed to the free side of the disc-shaped portion 10 of the mounting screw, for example, with an adhesive. When all the springs are clamped the protective sleeve 13 is mounted.

In use, the intervertebral disc prosthesis is inserted between two adjacent vertebrae to replace an intervertebral disc. The teeth 3 on the outer surface of the base plate and the top plate engage into the end plates of the adjacent vertebrae. During flexion and extension of the spine pressure is exerted onto or is relieved from one or several of the springs so that they are compressed or extended. Simultaneously the damping element 12 damps the purely axial motion. The springs 4 also provide rotational control, since the end sections 5a,5b are fixed which causes the loop-shaped sections 6 to be twisted to a certain degree when a torsional motion of the base plate and the top plate against each other occurs.

The Elasticity of the springs and the shape of the springs limits the Range of Motion (ROM) in flexion/extension to approximately +/−5° to protect neighbouring structures such as facet joints and ligaments against overloading. The substantial axial dampening, which is similar to that of a natural disc, limits axial compression forces to the physiological range and allows elastic interaction of the artificial disc and the facet joints (“lock facets”).

Modifications are possible. The shape of the base plate and the top plate can be adapted to more anatomically fit to the end plates of the vertebrae. Hence, it can be kidney-shaped or oval-shaped or otherwise shaped. The teeth can have another shape as that shown. For example, the teeth can be spikes or can have asymmetric shape. Furthermore, the outer surface of the base plate 1 and the top plate 2 can be roughened or coated for improving growth-in into the end plates.

The springs can be mounted by being screwed directly to the projection 1b,2b of the base plate 1 or the top plate 2 instead of clamping them.

The damping element 12 can be realized otherwise, for example by a hydrogel cushion or by other types of springs, such as a helical springs or disc springs.

The damping element 12 can also be omitted, although in this case the advantage of the axial damping is reduced. In addition, the protective sleeve can be omitted.

What is claimed is:

1. An intervertebral disc prosthesis comprising:
a base plate;
a top plate, and a central axis extending through a center of the base plate and the top plate;
a plurality of springs circumferentially spaced apart and arranged about the central axis between the base plate and the top plate, each spring having a loop-shaped section and two free ends;
a fastening device to connect the base plate to the top plate;
wherein one of the free ends of each spring is connected to the base plate and the other one of the free ends is connected to the top plate; and
wherein the loop-shaped section of each spring is directed away from the central axis.

2. An intervertebral disc prosthesis according to claim 1, wherein each of the springs is a substantially flat strip.

3. An intervertebral disc prosthesis according to claim 1, comprising a clamping device to clamp the free ends of the springs to the base plate and the top plate.

4. An intervertebral disc prosthesis according to claim 1, wherein the base plate and the top plate each have a projection coaxial with the central axis, the projections facing each other.

5. An intervertebral disc prosthesis according to claim 4, wherein the ends of the springs are clamped to the projections.

6. An intervertebral disc prosthesis according to claim 1, further comprising a damping element arranged between the base plate and the top plate.

7. An intervertebral disc prosthesis according to claim 1, wherein the springs are made from a material exhibiting super-elasticity.

8. An intervertebral disc prosthesis according to claim 1, further comprising a flexible protection sleeve arranged between the base plate and the top plate.

9. An intervertebral disc prosthesis according to claim 1, wherein the sides of the base plate and the top plate which are opposite to the springs comprise an engagement structure for engagement with an adjacent end plate of a vertebra.

10. An intervertebral disc prosthesis according to claim 1, wherein the free ends are adjacent the loop-shaped section.

11. An intervertebral disc prosthesis according to claim 7, wherein the springs comprise a shape memory alloy.

12. An intervertebral disc prosthesis according to claim 6, wherein the damping element is an elastomer.

13. An intervertebral disc prosthesis according to claim 1, wherein the fastening device comprises a damping element, and wherein the base plate is coupled to the top plate with the damping element.

14. An intervertebral disc prosthesis according to claim 1, wherein the fastening device is coaxial with the central axis.

15. An intervertebral disc prosthesis according to claim 1, wherein the fastening device comprises a clamping device to clamp the free ends of the springs to the base plate and the top plate.

16. A method of implanting an intervertebral prosthesis comprising a base plate, a top plate, and a central axis extending through a center of the base plate and the top plate, a plurality of springs circumferentially spaced apart and arranged about the central axis between the base plate and the top plate, each spring having a loop-shaped section and two free ends, and a fastening device to connect the base plate to the top plate, wherein one of the free ends of each spring is connected to the base plate and the other one of the free ends is connected to the top plate, wherein the loop-shaped section is directed away from the central axis, the method comprising:
compressing the prosthesis for insertion between two adjacent vertebrae;
inserting the prosthesis between the two adjacent vertebrae;
wherein the top plate and the base plate engage a corresponding vertebrae;
wherein each of the springs is configured to any one of compress and expand responsive to movement of the base plate relative to the top plate.

17. The method of claim 16, wherein the movement of the base plate relative to the top plate is damped with a damping element.

18. The method of claim 16, wherein the movement of the base plate relative to the top plate comprises rotation of the base plate relative to the top plate about the central axis.

19. A method of assembling an intervertebral prosthesis comprising a base plate, a top plate, and a central axis extending through a center of the base plate and the top plate, a plurality of springs circumferentially spaced apart and arranged about the central axis between the base plate and the top plate, each spring having a loop-shaped section and two free ends, and a fastening device to connect the base plate to the top plate, wherein one of the free ends of each spring is connected to the base plate and the other one of the free ends is connected to the top plate, wherein the loop-shaped section is directed away from the central axis, the method comprising:
arranging the at least two springs between the base plate and the top plate, the loop-shaped section of each spring being directed away from the central axis; and
connecting the base plate to the top plate with the fastening device, the connecting of the base plate to the top plate connecting one of the free ends of each spring to the base plate and the other one of the free ends of each spring to the top plate.

20. The method of claim 19, further comprising placing a damping element between the base plate and the top plate.

21. The method of claim 19, wherein the connecting of the springs to the base plate and the top plate comprises clamping the free ends of the springs to the base plate and the top plate, respectively, with the fastening device.

22. The method of claim 19, further comprising placing a flexible protection sleeve between the base plate and the top plate.
